(19) United States
(12) Patent Application Publication
(10) Pub. No.: US 2014/0065601 A1
(43) Pub. Date: Mar. 6, 2014

(54) METHOD AND KIT FOR ESTIMATING HUMAN IMMUNODEFICIENCY VIRUS (HIV) INCIDENCE

(71) Applicant: Tamar Jehuda-Cohen, Moshav Gimzo (IL)

(72) Inventor: Tamar Jehuda-Cohen, Moshav Gimzo (IL)

(21) Appl. No.: 14/006,688

(22) PCT Filed: Mar. 22, 2012
(86) PCT No.: PCT/IL12/50101

§ 371 Date: Nov. 6, 2013

Related U.S. Application Data
(60) Provisional application No. 61/466,892, filed on Mar. 23, 2011.

(51) Int. Cl. G01N 33/569

(52) U.S. Cl. CPC G01N 33/56983
USPC 435/5

(57) ABSTRACT

The present invention provides a method for determining the incidence of human immunodeficiency virus (HIV) infections in a population comprising comparing the anti-HIV antibody levels of in vitro stimulated tissue samples to those of un-stimulated tissue samples from individual members of said population and related kits. The present invention also provides a method of determining the distribution of recent, non-recent, and late stage human immunodeficiency virus (HIV) infections in a population comprising comparing the in vitro stimulated anti-HIV immunoreactivity and un-stimulated anti-HIV immunoreactivity in tissue samples from individual members of said population and related kits.

### FIELD OF THE INVENTION

The present invention provides a method for determining the incidence of human immunodeficiency virus (HIV) infections in a population comprising comparing the anti-HIV antibody levels of in vitro stimulated tissue samples to those of un-stimulated tissue samples from individual members of said population and related kits. The present invention also provides a method of determining the distribution of recent, non-recent, and late stage human immunodeficiency virus (HIV) infections in a population comprising comparing the in vitro stimulated anti-HIV immunoreactivity and un-stimulated anti-HIV immunoreactivity in tissue samples from individual members of said population and related kits.

### BACKGROUND OF THE INVENTION

Incidence (the rate at which new infections occur in a population) provides a more direct and current indication of the state of the HIV epidemic than does prevalence (the fraction of a population in an infected state at a point in time). Incidence measures provide invaluable information for assessing outbreaks, planning studies and targeting and assessing interventions.

In the past, the measurement of incidence through the direct observation of new infections in a population during the prospective follow-up of a cohort of initially seronegative individuals has been considered the ‘gold standard’ for incidence estimation. However, cohort studies are costly, logistically difficult to set-up and maintain, and results are prone to bias from unrepresentative recruitment and attrition of subjects.

Tests for Recent Infection (TRIs) therefore provide an attractive means of estimating incidence without the need for prospective follow-up. TRIs classify infections as recently or non-recently acquired, based on the results of laboratory tests that quantify biomarkers which evolve with time after infection, sometimes supplemented by clinical information. The prevalence of the TRI-defined ‘recent infections’ is estimated by applying the TRI in a cross-sectional survey of the population of interest. However, tests for recent HIV infection have traditionally been based on antibody avidity, proportion or titre, for which high false recent rates (ε) or low recency durations (ω) have hindered incidence estimation. Therefore, a new and better way of calculating incidence of new HIV infections is needed in the art.

The classification of infections by a TRI is usually based on measured biomarkers. One challenge is that evolution of these biomarkers within infected individuals exhibit inter-subject variability. In some cases, the state of recent infection is too transient for the population proportion to be estimated with good statistical power in studies with feasible samples sizes. In addition, there are often many individuals who remain classified as recently infected indefinitely or for very long periods, or who revert to a recent classification during end stage disease or under the influence of Anti-retroviral Treatment. Even though this phenomenon of ‘falsely recent’ infections may be explicitly accounted for without introducing bias in principle, there is considerable loss of statistical power when estimating the proportion of ‘truly recent’ infections for incidence estimation.

TRIs thus far proposed (such as detuned ELISAs, the BED assay and avidity assays) all crucially rely on measurements of antibody titre, avidity or HIV-specific proportion. However, these TRIs appear to be plagued by an unsatisfactory trade-off between the transient state of recent infection and false recent infections. In summary, for a TRI to be of utility in incidence estimation, the Mean Recency Duration (mean time spent in the state of recent infection) should be large, while the False Recent Rate (the proportion of long-infected individuals who remain in the TRI-defined state of recent infection) should be small. This invention addresses the need for a TRI meeting those criteria.

### SUMMARY OF THE INVENTION

In one embodiment, the present invention provides a method of determining the incidence of human immunodeficiency virus (HIV) infections in a population comprising the steps of: a) obtaining tissue samples from a representative number of subjects in said population; b) separating a first aliquot of each of said tissue samples for later determination of the initial anti-HIV antibody level; c) stimulating a second aliquot of each of said tissue samples to produce anti-HIV antibodies in vitro; d) determining the anti-HIV antibody level in said first and second aliquots of each of said tissue samples; e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample; f) determining if the SI obtained in step (e) for each sample is above a pre-determined threshold value, wherein a value below said threshold indicates that the sample was not recently infected and a value above said threshold indicates that the sample was recently infected; and g) calculating the mean number of recently infected samples divided by the product of the number of samples and the Mean Recency Duration for said threshold, thereby determining the incidence of HIV infections in said population.

In another embodiment, the present invention provides a kit for determining the incidence of human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific, and instructions for use.

In another embodiment, the present invention provides a method of determining the distribution of recent, non-recent, and late stage human immunodeficiency virus (HIV) infections in a population comprising the steps of: a) obtaining tissue samples from a representative number of subjects in said population; b) separating a first aliquot of each of said tissue samples for subsequent determination of the initial anti-HIV antibody level; c) stimulating a second aliquot of each of said tissue samples to produce anti-HIV antibodies in vitro; d) determining the anti-HIV antibody level in said first and second aliquots of each of said tissue samples; e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample; f) plotting the values of the SI obtained in step (e) for all samples to determine the distribution of recent, non recent, and late-stage infections, wherein samples with an SI value above a pre-determined threshold value have a recent infection, samples with an SI value of approximately said pre-determined threshold value have a non-recent infection, and seropositive samples with an SI value of less than said pre-determined threshold value have a late infection, thereby determining the distribution of recent, non-recent, and late stage HIV infections in said population.

In another embodiment, the present invention provides a kit for determining the distribution of recent, non-recent, and late stage human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific antibodies, and instructions for use.

### BRIEF DESCRIPTION OF THE DRAWINGS

FIG. 1: Stimulated and Un-stimulated Antibody Levels. Blood specimens incubated in stimulation media (Stimulated) have higher antibody levels compared to control blood specimens (Un-stimulated) after seroconversion. The increased antibody levels in the stimulated sample fade with time after seroconversion, and reverses at late stages of the infection.

FIG. 2: Graph of the Stimulation Index in a high risk population, with high incidence rates. A high proportion of the samples have a Stimulation Index (SI) of greater than 1.5, which is consistent with independent reports of high incidence rates in this population.

FIG. 3: Graph of the Stimulation Index in two high prevalence populations, with no new infections. None of the samples in either population A (A) or population B (B) have a Stimulation Index (SI) of greater than 1.2, which is consistent with independent reports of all the infections in these populations being non-recent (i.e. long term) ones. Also, no cases had an increased SI at the end stages as happens with other assays, where the late stages give similar values to the early ones, thus causing high levels of ‘false recent’. In fact, the SI at late stage decreases, as shown in FIG. 1.

FIG. 4: Stimulation Index (SI) Distribution. A sample distribution of recent, non-recent, and late stage infections in a population in which the infection is characterized by a long asymptomatic period.

FIG. 5: Stimulation Index (SI) Distribution for High Incidence Population. A sample distribution of recent and non-recent infections in a population with a high incidence of recent infections.

FIG. 6: Stimulation Index (SI) Distribution for a Population with Long-Term Infections. A sample distribution of non-recent and late stage infections in a population.

FIG. 7: Stimulation Index (SI) Distribution. A sample distribution of recent, non-recent, and late stage infections in a population in which the infection is characterized by a short asymptomatic period.

FIG. 8: Actual Stimulation Index (SI) Distribution for a Population with Long-Term Infections. A distribution of SI values from cross sectional data of all seropositives, from a first Chinese population with exclusively long-term infections.

FIG. 9: Actual Stimulation Index (SI) Distribution for a Population with Long-Term Infections. A distribution of SI values from cross sectional data of all seropositives, from a second Chinese population with exclusively long-term infections.

FIG. 10: Actual Stimulation Index (SI) Distribution for High Incidence Population. A distribution of SI values from cross sectional data of all seropositives, from a third Chinese population with a very high incidence rate.

FIG. 11: Actual Stimulation Index (SI) Distribution for High Incidence Population. A distribution of SI values from cross sectional data of all seropositives, from a Hungarian population with a very high incidence rate.

### DETAILED DESCRIPTION OF THE PRESENT INVENTION

In one embodiment, the present invention provides a method for determining the incidence of human immunodeficiency virus (HIV) infections in a population comprising comparing the anti-HIV antibody levels of in vitro stimulated tissue samples to those of un-stimulated tissue samples from individual members of said population and related kits. The present invention also provides a method of determining the distribution of recent, non-recent, and late stage human immunodeficiency virus (HIV) infections in a population comprising comparing the in vitro stimulated anti-HIV immunoreactivity and un-stimulated anti-HIV immunoreactivity in tissue samples from individual members of said population and related kits.

In one embodiment, the present invention provides a method of determining the incidence of human immunodeficiency virus (HIV) infections in a population comprising the steps of: (a) obtaining tissue samples from a representative number of subjects in said population; (b) separating a first aliquot of each of said tissue samples for later determination of the initial anti-HIV antibody level; (c) stimulating a second aliquot of each of said tissue samples to produce anti-HIV antibodies in vitro; (d) determining the anti-HIV antibody level in said first and second aliquots of each of said tissue samples; (e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample; (f) determining if the SI obtained in step (e) for each sample is above a pre-determined threshold value, wherein a value below said threshold indicates that the sample was not recently infected and a value above said threshold indicates that the sample was recently infected; and (g) calculating the mean number of recently infected samples divided by the product of the number of samples and the Mean Recency Duration for said threshold, thereby determining the incidence of HIV infections in said population.

In one embodiment, detectable anti-microbial antibody level in said first aliquot is an indication that a subject is seropositive. In one embodiment, the present invention provides methods of determining the incidence of new microbial infections in a population. In another embodiment, the present invention provides methods of determining the incidence of recent microbial infections in a population. In one embodiment, the present invention provides methods of determining the frequency of new microbial infections in a population.

In one embodiment, the present invention provides a method of determining the incidence of new microbial infections in a population comprising the steps of a) determining the anti-microbial antibody level in a first aliquot of a set of tissue samples from said population, wherein a detectable anti-microbial antibody level indicates that a sample is seropositive; b) determining the anti-microbial antibody level in a second aliquot from said tissue samples, said second aliquot having been stimulated to produce anti-microbial antibodies in vitro; and c) dividing a value representing the anti-microbial antibody level obtained in step (a) by a value representing the stimulated anti-microbial antibody level obtained in step (b) for each sample, wherein the mean number of samples with a value from step (c) that is higher than a pre-determined threshold divided by the total number of samples having a detectable level of anti-microbial antibody in step (b) and multiplied by Mean Recency Duration for said threshold provides a measure of the incidence of new microbial infections in said population.

In one embodiment, the present invention provides a method of determining the incidence of new microbial infections in a population comprising the steps of: (a) obtaining tissue samples from a representative number of subjects in said population; (b) determining the anti-microbial antibody level in a first aliquot of each of said tissue samples, wherein a detectable anti-microbial antibody level indicates that a sample is seropositive; (c) stimulating a second aliquot of each of said tissue samples from seropositive samples to produce anti-microbial antibodies in vitro and determining the anti-microbial antibody level in said second aliquot of each of said tissue samples; (d) dividing a value representing the stimulated anti-microbial antibody level obtained in step (c) by a value representing the anti-microbial antibody level obtained in step (b) for each sample; (e) determining if the quotient obtained in step (d) for each sample is above a pre-determined threshold value, wherein a value below said threshold indicates that the sample was not recently infected and a value above said threshold indicates that the sample was recently infected; (f) calculating the mean number of recently infected samples divided by the product of the number of seropositive samples and the Mean Recency Duration for said threshold, thereby determining the incidence of new microbial infections in said population.

In another embodiment, the present invention provides a method of determining the distribution of recent, non-recent, and late stage human immunodeficiency virus (HIV) infections in a population comprising the steps of: (a) obtaining tissue samples from a representative number of subjects in said population; (b) separating a first aliquot of each of said tissue samples for subsequent determination of the initial anti-HIV antibody level; (c) stimulating a second aliquot of each of said tissue samples to produce anti-HIV antibodies in vitro; (d) determining the anti-HIV antibody level in said first and second aliquots of each of said tissue samples; (e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample; (f) plotting the values of the SI obtained in step (e) for all samples to determine the distribution of recent, non recent, and late-stage infections in said population, wherein samples with an SI value above a pre-determined threshold value have a recent infection, samples with an SI value of approximately said pre-determined threshold value have a non-recent infection, and seropositive samples with an SI value of less than said pre-determined threshold value have a late infection, thereby determining the distribution of recent, non-recent, and late stage HIV infections in said population.

In another embodiment, the present invention provides a method of determining the epidemiological state of human immunodeficiency virus (HIV) infections in a population comprising the steps of: (a) obtaining tissue samples from a representative number of subjects in said population; (b) separating a first aliquot of each of said tissue samples for subsequent determination of the initial anti-HIV antibody level; (c) stimulating a second aliquot of each of said tissue samples to produce anti-HIV antibodies in vitro; (d) determining the anti-HIV antibody level in said first and second aliquots of each of said tissue samples; (e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample; (f) plotting the values of the SI obtained in step (e) for all samples to determine the distribution of recent, non recent, and late-stage infections in said population, wherein samples with an SI value above a pre-determined threshold value have a recent infection, samples with an SI value of approximately said pre-determined threshold value have a non-recent infection, and seropositive samples with an SI value of less than said pre-determined threshold value have a late infection, thereby determining the distribution of recent, non-recent, and late stage HIV infections in said population and thus the epidemiological state of HIV infection in said population.

In another embodiment, the methods of the present invention may be used to determine the epidemiological state of an infection in a population. In one embodiment, the epidemiological state is the relationship between the different stages of the infection in a specified population.

In another embodiment, the present invention provides a method of determining the distribution of recent, non-recent, and late stage microbial infections in a population comprising the steps of: (a) obtaining tissue samples from a representative number of subjects in said population; (b) determining the anti-microbial antibody level in a first aliquot of each of said tissue samples, wherein a detectable anti-microbial antibody level indicates that a sample is seropositive; (c) stimulating a second aliquot of each of said tissue samples from seropositive samples to produce anti-microbial antibodies in vitro and determining the anti-microbial antibody level in said second aliquot of each of said tissue samples; (d) dividing a value representing the stimulated anti-microbial antibody level obtained in step (c) by a value representing the anti-microbial antibody level obtained in step (b) for each sample; (e) plotting the values of the quotient obtained in step (d) for all said seropositive samples to determine the distribution of recent, non recent, and late-stage infections in said population, wherein seropositive samples with a quotient value of greater than a pre-determined threshold value have a recent infection, seropositive samples with an quotient value of approximately the pre-determined threshold value have a non-recent infection, and seropositive samples with a quotient value of lower than the pre-determined threshold value have a late infection, thereby determining the distribution of recent, non-recent, and late stage microbial infections in said population.

In one embodiment, the method further comprises the step of calculating the ratio of recent, non-recent, and late stage HIV infections to total HIV infections in said population. In another embodiment, the method further comprises the step of calculating the area under the curve (AUC) of the plot obtained in step (f), wherein a larger AUC above the threshold indicates more recent HIV infections and a larger AUC under the threshold indicates more late-stage HIV infections in the population. In one embodiment, the AUC measurement takes into account how recent an infection is, i.e. how high the SI is rather than only considering whether the SI exceeds a pre-determined threshold.

In one embodiment, the present invention provides a tissue sample from one or more subjects for evaluation. In one embodiment, the tissue sample is a blood sample. In another embodiment, the tissue sample is a whole blood sample. In another embodiment, the sample comprises cells in blood or saliva from said subject. In another embodiment, the tissue sample is a cheek or tongue swab. In another embodiment, the tissue sample is a biopsy (e.g. lymph node, liver, etc).

As used herein, the term “whole blood” means blood collected with heparin, EDTA, citrate, or any other substance that prevents coagulation and clotting. The term whole blood as used herein also includes blood collected from an animal or human with heparin, ethylenediaminetetraacetate, citrate, or any other substance that prevents coagulation and clotting. “Whole blood” can also mean blood wherein the red blood cells have been lysed while maintaining the viability of the remaining white blood cells.

The term “sample” includes samples present in an individual as well as samples obtained or derived from the individual.

In one embodiment, the methods of the present invention comprise the step of determining if the SI of each sample is above a threshold value. In one embodiment, a value below said threshold indicates that the sample was not recently infected and a value above said threshold indicates that the sample was recently infected. In another embodiment, a value below said threshold indicates that the sample is from a source or subject that was not recently infected and a value above said threshold indicates that the sample is from a source or subject that was recently infected.

In one embodiment, a quotient value of greater than 1.2 indicates a recent infection. In one embodiment, a quotient value of lower than 1.0 indicates a late infection. In another embodiment, a quotient value of lower than 0.9 indicates a late infection. In another embodiment, a quotient value of lower than 0.8 indicates a late infection. In another embodiment, a quotient value of approximately 1.0 indicates a non-recent infection. In another embodiment, a quotient value of between 0.9 and 1.1 indicates a non-recent infection. In another embodiment, a quotient value of between 0.8 and 1.2 indicates a non-recent infection.

In one embodiment, a distribution skewed toward quotient values above the pre-determined threshold is an indication of a population with a high incidence or, in another embodiment, a population having many recent infections. In another embodiment, a distribution skewed toward quotient values below the pre-determined threshold is an indication of a population having many late stage infections.

In another embodiment, the present invention provides a method of evaluating the efficacy of a strategy for the prevention of the spread of an infection in a population comprising the step of comparing the distribution of recent infections, non-recent infections, and late stage infections in a particular population to a prior distribution of infections in said population, wherein a shift of the distribution away from new infections in a population indicates a successful prevention strategy. In another embodiment, the present invention provides a method of evaluating the efficacy of a treatment strategy for an infection in a population comprising the step of comparing the distribution of recent infections, non-recent infections, and late stage infections in a particular population to a prior distribution of infections in said population, wherein a shift of the distribution away from late stage infections and to the non-recent (long-term) range indicates a successful treatment strategy for the population. In another embodiment a successful treatment strategy is further characterized by a decrease in samples with new infections in the population, which may be determined as described herein. In one embodiment, the prior distribution of infections in the population was from one year earlier. In another embodiment, the prior distribution of infections in the population was from two years earlier. In another embodiment, the prior distribution of infections in the population was from five years earlier.

In another embodiment, the success of a treatment or prevention strategy for stopping the spread of infection in a population is evaluated by determining a current distribution of infections to an expected distribution of infections, which in one embodiment, relies on epidemiological data from other populations with characteristics in common that are relevant to spread of the infection in a population. In another embodiment, the epidemiological data collected at a single time point by plotting the distribution of stimulation indices suffices to provide epidemiological data on the population.

In another embodiment, the present invention provides a method of determining the incidence of new viral infections in a population comprising the steps of a) determining the anti-virus antibody level in a first aliquot of a set of blood samples from said population, wherein a detectable anti-viral antibody level indicates that a sample is seropositive; b) determining the anti-virus antibody level in a second aliquot from said blood samples, said second aliquot having been stimulated to produce anti-viral antibodies in vitro; and c) dividing a value representing the anti-virus antibody level obtained in step (a) by a value representing the stimulated anti-virus antibody level obtained in step (b) for each sample, wherein the mean number of samples with a value from step (c) that is higher than a pre-determined threshold divided by the total number of samples having a detectable level of anti-virus antibody in step (b) and multiplied by Mean Recency Duration for said threshold provides a measure of the incidence of new viral infections in said population.

In one embodiment, the method further comprises the step of obtaining or collecting a blood sample from said population prior to step (a).

In one embodiment, the viral infection is a retroviral infection. In one embodiment, the retrovirus is HIV. In another embodiment, it is a retrovirus. In one embodiment, the retrovirus is Alpharetrovirus, which in one embodiment is an Avian leukosis virus or a Rous sarcoma virus. In another embodiment, the retrovirus is a betaretrovirus, which in one embodiment, is a mouse mammary tumour virus. In another embodiment, the retrovirus is a gammaretrovirus, which in one embodiment, is a murine leukemia virus or feline leukemia virus. In another embodiment, the retrovirus is a deltaretrovirus which in one embodiment, is a bovine leukemia virus or the cancer-causing Human T-lymphotropic virus (HTLV), which in one embodiment, is HTLV-1, and in another embodiment, it is HTLV-2. In another embodiment, the retrovirus is a epsilonretrovirus, which in one embodiment, is a Walleye dermal sarcoma virus. In another embodiment, the retrovirus is a lentivirus, which in one embodiment, is a human immunodeficiency virus 1, Simian immunodeficiency virus, or Feline immunodeficiency virus. In another embodiment, the retrovirus is a spumavirus, which in one embodiment, is a simian foamy virus. In another embodiment, the retrovirus is a hepatitis C virus (HCV). In another embodiment, the retrovirus is a hepatitis E virus (HEV). In another embodiment, the retrovirus is a hepatitis D virus (HDV).

In one embodiment, a virus related to the methods and kits of the present invention is xenotropic murine leukemia virus (XMRV). In another embodiment, the virus is hepatitis A virus (HAV). In another embodiment, the virus is hepatitis B virus (HBV). In another embodiment, the virus is hepatitis C virus (HCV). In another embodiment, the virus is hepatitis D virus (HDV). In another embodiment, the virus is hepatitis E virus (HEV). In another embodiment, the virus is Human T-lymphotrophic virus-1 (HTLV-1). In another embodiment, the virus is any combination of the viruses disclosed hereinabove. In another embodiment, the virus is hepatitis B virus (HBV), hepatitis C virus (HCV), or hepatitis E (HEV) virus.

In another embodiment the virus is human immunodeficiency virus (HIV). In one embodiment, the HIV is HIV-1. In another embodiment, the HIV is HIV-2. In another embodiment, the HIV is HIV-0.

In one embodiment, the methods of the present invention may be used to determine the incidence of new infections. In one embodiment, “incidence” is the frequency with which new infections appear in a particular population or area. In one embodiment, incidence is the number of newly diagnosed cases during a specific time period.

In one embodiment, the present invention provides a method of determining the prevalence of a microbial infection in a population comprising the steps of: (a) obtaining tissue samples from a representative number of subjects in said population; (b) stimulating an aliquot of each of said tissue samples to produce anti-microbial antibodies in vitro; (c) determining the anti-microbial antibody level in said tissue samples; (d) comparing the number of samples with detectable anti-microbial antibody levels to the number of samples with no detectable anti-microbial antibody levels, thereby determining the prevalence of a microbial infection in said population.

In one embodiment, the infection is a chronic infection. In one embodiment, a chronic infection is characterized by the continued presence of the infectious microbe following the initial infection and can include chronic or recurrent disease. In one embodiment, a microbe is a microscopic living organism, such as a bacterium, fungus, protozoan or virus.

In one embodiment, the chronic infection is a microbial infection. In another embodiment, the chronic infection is a viral infection, which in one embodiment, is a retroviral infection. In another embodiment, the chronic infection is a bacterial infection, which in one embodiment is a tuberculosis (TB) infection. In one embodiment, the chronic viral infection is a measles (paramyxovirus), hepatitis, or infectious mononucleosis infection. In one embodiment, the infection is a herpesvirus infection, which in one embodiment, is a cytomegalovirus (CMV) infection. In another embodiment, the herpesvirus infection is an Epstein-Barr Virus infection. In another embodiment, the herpesvirus infection is a herpes simplex virus (HSV) infection, which in one embodiment, is an HSV-1 or HSV-2 infection, or, in another embodiment, the herpesvirus infection is a varicella-zoster virus (VZV),

In another embodiment, the present invention provides a method of determining the incidence of new human immunodeficiency virus (HIV) infections in a population comprising the steps of a) determining the anti-HIV antibody level in a first aliquot of a set of blood samples from said population, wherein a detectable anti-HIV antibody level indicates that a sample is seropositive; b) determining the anti-HIV antibody level in a second aliquot from said blood samples, said second aliquot having been stimulated to produce anti-HIV antibodies in vitro; and c) dividing a value representing the stimulated anti-HIV antibody level obtained in step (a) by a value representing the anti-HIV antibody level obtained in step (b) for each sample, wherein the mean number of samples with a value from step (c) that is higher than a pre-determined threshold value divided by the total number of samples having a detectable level of anti-HIV antibody in step (b) and multiplied by the Mean Recency Duration for said threshold provides a measure of the incidence of new HIV infections in said population.

In one embodiment, the methods of the present invention involve comparing a value, such as an SI value to a pre-determined threshold value. In one embodiment, the threshold value is determined based on the SI data of the population tested. In one embodiment, the threshold value is determined based on the data of the specific population set tested, as is known in the art. In another embodiment, the threshold valued is determined based on a different population with similar geographic, cultural, medical, or other characteristics.

In another embodiment, the present invention provides a method of determining the incidence of HIV infection in a population, the method comprising determining the ratio of in vitro stimulated anti-HIV immunoreactivity and un-stimulated anti-HIV immunoreactivity in blood samples from individual members of said population, wherein the proportion of samples having a ratio that is higher than a pre-selected threshold ratio is a measure of the incidence of HIV in said population. In one embodiment, said pre-selected threshold ratio is 1.1.

In one embodiment, the methods of the present invention are used for epidemiological studies. In one embodiment, epidemiological studies involve the distribution of determinants of health-related states (such as comparative antibody levels with and without stimulation) in a population and the use of this information to address health-related epidemiological problems.

In another embodiment, the present invention provides a method of determining the incidence of new human immunodeficiency virus (HIV) infections in a population comprising the steps of: (a) obtaining blood samples from a representative number of subjects in said population; (b) determining the anti-HIV antibody level in a first aliquot of each of said blood samples, wherein a detectable anti-HIV antibody level indicates that a sample is seropositive; (c) stimulating a second aliquot of each of said blood samples to produce anti-HIV antibodies in vitro and determining the anti-HIV antibody level in said second aliquot of each of said blood samples; (d) dividing a value representing the HIV antibody level obtained in step (b) by a value representing the stimulated HIV antibody level obtained in step (c) for each sample; (e) determining if the quotient obtained in step (d) for each sample is above a pre-determined threshold value, wherein a value below said threshold indicates that the sample was not recently infected and a value above said threshold indicates that the sample was recently infected; and (f) calculating the mean number of recently infected samples divided by the number of seropositive samples and multiplied by the Mean Recency Duration for said threshold, thereby determining the incidence of new HIV infections in said population.

In one embodiment, the present invention provides a method of determining the incidence of new HIV infections in a “population” using the methods described herein. In one embodiment, the population is a population of interest. In another embodiment, the population is a population known in the art. In one embodiment, the word “population” shall be taken to mean a group of people according to their race, country of origin, socio-economic condition, sex, sexual orientation, age, religion, employment, health, etc. In one embodiment, the population is a population that is vulnerable to developing a retrovirus infection, which in one embodiment, is HIV. In one embodiment, the population is defined by a particular behavior, which in one embodiment, is intravenous drug use, homosexual activity, bisexual activity, sexual activity with multiple partners, prostitution, receipt of blood transfusions, or a combination thereof. In another embodiment, the population is defined by living or having visited or travelled though a particular geographic location, which in one embodiment, is a continent, a region, a state, or a city. In another embodiment, the population is defined by a particular medical status, which in one embodiment is a hemophiliac, a subject with one or more sexually transmitted diseases, etc. In one embodiment, the population is derived from a particular subgroup of the populations described hereinabove, such as, in one embodiment, intravenous drug users from China.

In one embodiment, formulas, tables, and power function charts known in the art may be used to statistically determine what is a representative number of subjects for the population. As will be apparent to those skilled in the art of epidemiology, it is not necessary to assay every member of a population to obtain the incidence of a particular character. Accordingly, a sufficient number of individuals of the population to provide a statistically significant estimate of the population is tested, and as a consequence, the actual numbers to be tested is readily determined without undue experimentation. In one embodiment, the individuals tested are randomly selected from the population.

In one embodiment, the level of anti-microbial or anti-virus antibody in a non-activated blood sample versus an activated blood sample is the Stimulation Index (SI) value. In one embodiment, the SI values will be measured with varying sensitivity or amplitude depending on the detection system used. Thus, the SI values considered as “elevated” in accordance with the present invention will depend upon the precise procedure utilized. The SI values can be tested against samples obtained from individuals known to be recently infected with HIV and compared with similar samples obtained from individuals who have an established HIV infection such as, but not limited to, individuals who are known to have been infected for at least 1 year or so. Upon comparison of the results, a suitable SI value can be determined which readily distinguishes a recent infection as defined herein from an established infection. Assay variation can be controlled by using the value from a standard set of sample pairs. A skilled artisan could readily use standard techniques to determine a suitable SI threshold value when using any of a variety of methods of detecting immunoreactivity to a retroviral antigen. In one embodiment, the methods of the present invention further comprise the step of determining or estimating a threshold SI value, wherein a value below said threshold indicates that the sample was not recently infected and a value above said threshold indicates that the sample was recently infected. In one embodiment, the threshold SI value is the pre-determined threshold used in the methods of the invention.

In one embodiment, the methods of the present invention comprise calculating the mean number of recently infected samples divided by the number of seropositive samples and multiplied by the Mean Recency Duration for said threshold. In one embodiment, the methods of the present invention comprise calculating the mean number of recently infected samples divided by the product of the number of seropositive samples and the Mean Recency Duration for said threshold. In another embodiment, (the mean number of recently infected samples/the number of seropositive samples) x the Mean Recency Duration for the threshold=a measure of the incidence of new viral infections in said population.

In another embodiment, the methods of the present invention comprise calculating the mean number of recently infected samples divided by the number of samples and multiplied by the Mean Recency Duration for said threshold. In one embodiment, the methods of the present invention comprise calculating the mean number of recently infected samples divided by the product of the number of samples and the Mean Recency Duration for said threshold. In another embodiment, (the mean number of recently infected samples/the number of samples)×the Mean Recency Duration for the threshold=a measure of the incidence of new viral infections in said population.

In one embodiment, the “Mean Recency Duration” is a pre-determined time period defined as recent for said threshold. In one embodiment, “recency” is described by the “Mean Recency Duration”, which in one embodiment, is the average time period after infection in which there is an SI greater than the threshold value described hereinabove. For example and in one embodiment, the Mean Recency Duration may be 1 year for a SI threshold value of 1.2, which would mean that subjects with an SI value of 1.2 or higher were likely infected within the last year. In one embodiment, the methods of the present invention further comprise the step of determining or estimating the Mean Recency Duration for a specific SI threshold.

In one embodiment, the present invention provides a method of determining the incidence of “new” microbial or viral infections in a population. In one embodiment, “new” microbial or viral infections are understood to be “recent” viral infections. As described hereinabove, recent infections are determined based on a pre-determined SI value and Mean Recency Duration value, which in turn are based on analysis of an initial population in which the recency of infection is known, as is understood by one of skilled in the art.

In one embodiment, detecting “immunoreactivity” comprises measuring antigen-induced secretions by B cells and T cells, where in one embodiment, antibody, cytokine, lymphokine, or a combination thereof, are secreted.

In one embodiment, a sample of the present invention is obtained from a bodily fluid, such as fresh whole blood in which a single aliquot is activated and the rest of the sample is not activated, as described herein, or in another embodiment, a sample is a pair of plasma samples, in which one of the plasma pair was from activated and the other plasma pair was from not activated blood.

In some embodiments, the method comprises the steps of: (i) collecting a first assay sample prior to the incubation; (ii) measuring the level of antibodies in said first assay sample; (iii) collecting a second assay sample after the incubation; (iv) measuring the level of antibodies in said second assay sample; and (v) comparing the measurements of the levels of antibodies between said first and second assay samples.

In other embodiments, the method comprises the steps of: (i) collecting a first assay sample prior to the incubation step and storing said first assay sample; (ii) collecting a second assay sample after said incubation step; (iii) measuring the level of antibodies in said first and said second assay sample concurrently; and (iv) comparing the measurements of the levels of antibodies between said first and second assay samples.

In another embodiment, the methods of the present invention comprise the step of first determining the anti-HIV antibody level in the stimulated aliquot or sample, and if the anti-HIV antibody level in the stimulated aliquot is detectable, then the method comprises the step of determining the anti-HIV antibody level in the non-stimulated aliquot or sample.

In another embodiment, the methods of the present invention comprise the step of first determining the anti-HIV antibody level in the non-stimulated aliquot or sample, and if the anti-HIV antibody level in the non-stimulated aliquot is detectable, then the method comprises the step of determining the anti-HIV antibody level in the stimulated aliquot or sample.

In one embodiment, “concurrently” refers to running the first assay sample and second assay sample in the same antibody detecting assay on the same day, which, in one embodiment, provides more direct comparative data and provides less assay variation than assays run on separate days. In one embodiment, the first assay sample is stored until after the incubation and collection of the second sample. In one embodiment, the two samples (i.e. before and after incubating with the activator described herein) are taken from the same tube. In one embodiment, the first sample is from time 0 days and the second sample is from time Xdays, wherein X is the number of days that the sample is incubated with the activator or activators as described herein. In another embodiment the non-activated sample is collected at “time 0” and the other, activated one, is taken after a period of incubation with the activator, which in one embodiment is time 5 (i.e. after 5 days of activation).

In one embodiment, the tissue or blood sample is stimulated for one day. In another embodiment 2 days. In another embodiment, the tissue or blood sample is stimulated for 3 days. In another embodiment, the tissue or blood sample is stimulated for 4 days. In another embodiment, the tissue or blood sample is stimulated for 5 days. In another embodiment, the tissue or blood sample is stimulated for 6 days. In another embodiment, the tissue or blood sample is stimulated for 7 days. In another embodiment, the tissue or blood sample is stimulated for 3-5 days. In another embodiment, the tissue or blood sample is stimulated for 2-7 days.

In one embodiment, the plasma and stimulated-plasma are stored “properly”, which in one embodiment, is at a temperature of 4° C. (for short term storage of days), or in another embodiment, at a temperature of −20° C. or −80° C. (for long term storage of over a week), as is well known in the art. In one embodiment, the plasma may be stored for up to 2 days. In another embodiment, the plasma may be stored for up to 7 days. In another embodiment, the plasma may be stored for up to 14 days. In another embodiment, the plasma may be stored for up to 1 month. In another embodiment, the plasma may be stored for up to 6 months. In another embodiment, the plasma may be stored for up to 12 months. In another embodiment, the plasma may be stored for up to 24 months. In another embodiment, the plasma may be stored for up to 3 years. In another embodiment, the plasma may be stored for up to 5 years. In another embodiment, the plasma may be stored for up to 10 years. In another embodiment, the plasma may be stored for up to 20 years.

In one embodiment the HIV can be any strain or isolate. Preferably, the HIV is selected from the group consisting of HIV-1, HIV-2, or a combination thereof.

In one embodiment, the subject is a mammal, which in one embodiment, is a primate, which in one embodiment, is a human.

In one embodiment, the incidence of new HIV infections is determined using the methods of the present invention. In another embodiment, the incidence of new HIV infections is estimated using the methods of the present invention. In another embodiment, the incidence of new HIV infections is approximated using the methods of the present invention.

In one embodiment, the incidence of new HIV infections is determined using the methods of the present invention. In another embodiment, the prevalence of new HIV infections is determined using the methods of the present invention. In another embodiment, the percentage of new HIV infections is determined using the methods of the present invention. In another embodiment, the infection rate for HIV infections is determined using the methods of the present invention.

In one embodiment, a lower incidence of new infections in a population following one or more prevention programs, interventions, or strategies is a sign that the prevention programs, interventions, or strategies for preventing spread of a particular microbe or virus such as HIV are or were effective.

In one embodiment, a method of the present invention requires the determination of anti-microbial antibody levels in a tissue sample. In another embodiment, a method of the present invention requires the determination of anti-microbial antibody types in a tissue sample. In another embodiment, a method of the present invention requires the determination of anti-microbial antibody affinity in a tissue sample. In another embodiment, a method of the present invention requires the determination of anti-microbial antibody avidity in a tissue sample. All the above can be determined in both the stimulated and unstimulated sample, and the above, individually or in various combinations may be compared to one another.

In one embodiment, the tissue sample is a blood sample. In another embodiment, the tissue sample is obtained from the gum or cheek of the subject.

In one embodiment, a method of the present invention requires determining the anti-retroviral antibody level in an aliquot of a blood sample. In one embodiment, an “aliquot” is a portion of the total amount of a blood sample. In one embodiment, the aliquots used in the methods of the present invention are of equal volume or dilution. In one embodiment, duplicate blood samples are used in the methods of the present invention. In one embodiment, the first and second aliquots of a blood sample are portions of a single blood sample drawn from a single subject at a single time point.

In another embodiment, a single aliquot of the tissue or blood sample may be used to determine both “baseline” antibody levels and stimulated antibody levels, wherein a tissue sample, such as blood is drawn into a container comprising the activator described herein and the cells are sedimented (by regular G force, or by a short centrifugations at low speed). A small aliquot of the plasma supernatant is removed for later testing of the initial levels of HIV/HCV/retrovirus/virus/pathogen/microorganism. The rest is incubated with the activator for several days. The levels of the antibodies against HIV/HCV/retrovirus/virus/pathogen/microorganism for the aliquot removed at Time( ) is measured on the same assay with an aliquot of blood or tissue removed after the incubation. The two measurements are compared. In one embodiment, the delta is calculated, in another embodiment, the ratio of signals or levels, is calculated, in another embodiment, the ratio of IgM to IgG of antibodies against HIV/HCV/retrovirus/virus/pathogen/microorganism is calculated, etc.

In accordance with the present invention, a blood sample is drawn into a test tube, which in one embodiment, is a vacuum-tube, a bottle, a well (as part of a multi well plate or as a single well or plate) or a flask, containing an effective concentration of a solution of a activators (such as mitogens, cytokines, lymphokines, and combinations thereof as described hereinabove). The blood sample to be tested is cultured in vitro in the presence of any combination of activators of lymphocytes to achieve the same function.

In one embodiment, the step of determining anti-microbial antibody levels comprises performing an antibody assay on each aliquot of said blood samples. In one embodiment, an antibody assay comprises exposing each of said blood samples to a viral antigen thereby allowing an antigen-antibody immune complex to form and detecting said antigen-antibody immune complex. In one embodiment, detection of the antigen-antibody immune complex is semi-quantitative.

In one embodiment, said antigen is added to said culture to shorten the incubation time and/or to provide diagnosis in situ. In another embodiment, the antigen-antibody immune complex is detected on a solid phase support, carrier, or solid base, which in one embodiment, is a nitrocellulose strip, a set of labeled or colored beads, or any other carrier. In one embodiment, the carrier may comprise beads with different densities, sizes, labels, colors, fluorescence, as is known in the art.

In one embodiment, after incubation, an aliquot is taken from the supernatant and is then assayed for the presence of desired antibodies using standard standard Rapid ELISA, Western Blot analysis, a lateral flow, or an immunofluorescence assay, and/or any other antibody detection system, which in one embodiment is a Chemiluminescence, luminescence, or chip system. In one embodiment, the assay is an enzyme immunoassay (ETA) including enzyme-linked immunosorbent assay (ELISA), radioimmunopreciptation assay (RIPA), particle agglutination assay or immunofluorescence assay (IFA).

In one embodiment, the antibody assay is an enzyme linked immunosorbent assay, a blot, a chemi-illuminesense assay, a luminescence assay, or an immunofluorescence assay, a peptide-chip-array, or an antibody chip array. In one embodiment, the antibody assay is any semi-quantitative assay for HIV antibodies known in the art, total or specific.

If the sample is to be assayed at a later date, the supernatant fluid may be collected, frozen and stored.

General Techniques: Unless otherwise indicated, the immunological techniques utilized in the present invention are standard procedures, well known to those skilled in the art. Such techniques are described and explained throughout the literature in sources such as, Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

General Methods for Detecting an HIV Infection: Many techniques have been developed for detecting an HIV infection. At least some of these procedures are commercially available in “kit” form. Many of the techniques are generally described in HIV: A Practical Approach (Volume 1: Virology and Immunology. Ed. Jonathan Karn, IRL Press); AIDS Testing: A comprehensive guide to technical, medical, social, legal, and management issues (Ed. Gerald Schochetman and J. Richard George. 2.sup.nd Edition. Springer-Verlag, 1994); Gallo et al. (1986) and Mylonakis et al. (2000). An overview of at least some of these techniques is provided below. Furthermore, at least some of these techniques, including those of the claimed invention, can readily be adapted to be performed using nanocrystals such as those described in WO 00/27365, U.S. Pat. No. 6,207,392, Nolan and Sklar (2002), and Han et al. (2001).

Enzyme Immunoassay (EIA) or Enzyme-Linked Immunosorbent Assay (ELISA) Methodology: ELISA detection systems have been used routinely in EIAs for many years in the detection of HIV infection by showing the presence of anti-HIV antibodies. Furthermore, there are many licensed manufacturers of EIAs and ELISAs for detecting antibody to HIV. The sensitivity of third generation EIAs is close to 100 percent when any anti-HIV-antibody is present in peripheral blood. However, these assays cannot differentiate between the earliest stages of infection and established infection.

EIA methodology involves the following steps. HIV-antigens are purified from viral lysate, prepared by recombinant DNA technology or peptide synthesis and are coated onto the wells of microwell plates or onto other matrixes such as beads to form the “solid phase” of the assay. The serum of an individual is added to the well. Antibody, if present, reacts with the antigen, and the other well contents are then washed away. An indicator reagent consisting of an anti-human antibody bound to an enzyme or other detection system is added to the well. If the serum contained HIV-specific antibodies, these will remain attached to the solid-phase antigen, and the enzyme-conjugated anti-human antibody will attach to these antibodies and thus to the solid phase. Another washing step follows. If the individual's serum contains antibody to HIV, the enzyme remains attached through antibody to the solid phase and is available to catalyze a color-producing reaction when an appropriate substrate is added to the well. The color change is measured in a spectrophotometer. Absorbance values above a cut-off value calculated from control samples are considered reactive. Within the linear (or reactive) range of the assay, the absorbance values are directly related to the levels of antibodies in the tested sample.

This basic methodology has been adapted to encompass a wide variety of assay formats including, both antigen and antibody capture assays as well as antigen and antibody competition assays.

Immuno Transfers (Western Blots and other antigen blots): Blots are another form of EIA which have been commonly used for establishing the presence of true anti-HIV antibodies. Several commercially produced kits are available. Certain blots may be used in a semi-quantitative way.

Particle Agglutination Assays: Antigen or antibody labeled latex particles, sepharose, polyurethane microcapsules, colloidal gold or red blood cells have been employed to produce a wide range of immuno-agglutination assays. Particles can be obtained commercially with a large range of surface chemistries allowing for great flexibility when coupling them to either antibody or antigen. These techniques are typically used in rapid assay formats that are usually scored visually, but are also adapted to automation and semi-quantification. Immunofluorescence Assay (IFA)

The IFA for HIV-antibody is more technically demanding and more expensive than Western blots. Because virtually all the antigens present in an infected cell are available for reaction with the test specimen, it is a very sensitive assay. It is a procedure familiar to many laboratories because it is used for detecting antibodies to a wide variety of viral and bacterial antigens.

Basically, the technique involves the following steps. A suspension of a lymphocyte cell culture infected with HIV is placed on a microscope slide, air-dried, and fixed in acetone or methanol. Uninfected control cells are added to the suspension or put in separate spots on the slide to provide a means for detecting non-specific reactions (fixed slides can be made in large batches and stored frozen or desiccated.) Diluted test sera are added to the cell spots, the slide is washed, incubated again with fluorescein-conjugated anti-human globulin, washed again, and then inspected for fluorescein fluorescence using an ultraviolet microscope.

Typical localized fluorescence of infected cells occurs after reaction with positive sera. Little or no fluorescence occurs with negative sera. Non-specific reactions (such as those caused by antinuclear antibody) are recognized because of fluorescence in uninfected control cells.

Radioimmunoprecipitation: The radioimmunoprecipitation assay is used primarily in research. It is generally too technically demanding for routine use in clinical laboratories. Radioimmunoprecipitation is especially sensitive for antibodies to the higher molecular weight major envelope glycoproteins gp160 and gp120, which some Western blot techniques miss. The principle of RIPA involves competitive binding of radiolabeled antigen and unlabeled antigen to a high-affinity antibody. The antigen is generally labelled with a gamma-emitting isotope such as ¹²⁵I. The labelled antigen is mixed with antibody at a concentration that just saturates the antigen-binding sites of the antibody molecule, and then increasing amounts of unlabeled antigen of unknown concentration are added. The antibody does not distinguish labelled from unlabeled antigen, and so the two kinds of antigen compete for available binding sites on the antibody. With increasing concentrations of unlabeled antigen, more labelled antigen will be displaced from the binding sites. By measuring the amount of labelled antigen free in solution, it is possible to determine the concentration of unlabeled antigen.

In one embodiment, FDA approved HIV tests known in the art include, inter alia, Abbott HIVAB HIV-1/HIV-2 (rDNA) EIA, Abbott Laboratories, Abbott Park, IL; ABBOTT PRISM HIV O Plus assay, Abbott Laboratories, Abbott Park, Ill.; ARCHITECT HIV Ag/Ab Combo, Abbott Laboratories, Abbott Park, Ill.; HIVAB HIV-1 EIA, Abbott Laboratories, Abbott Park, Ill.; Abbott RealTime HIV-1 Amplification Kit, ABBOTT Molecular, Inc., Des Plaines, Ill.; Avioq HIV-1 Microelisa System, Avioq Inc., Rockville, Md.; Human Immunodeficiency Virus, Type 1 (HIV-1) Reverse Transcription (RT) Polymerase Chain Reaction (PCR) Assay, BioLife Plasma Services, L.P., Deerfield, Ill.; INSTI™ HIV-1 Antibody Test Kit, bioLytical Laboratories Inc., British Columbia, Canada V6V 2X7; GS rLAV EIA, Bio-Rad Laboratories Redmond, Wash.; Bio-Rad GS HIV Ag/Ab Combo EIA, Bio-Rad Laboratories, Redmond, Wash.; GS HIV-1 Western Blot, Bio-Rad Laboratories, Redmond, Wash.; GS HIV-1/HIV-2 Plus O EIA, Bio-Rad Laboratories, Redmond, WA; GS HIV-2 EIA, Bio-Rad Laboratories, Redmond, Wash.; Multispot HIV-1/HIV-2 Rapid Test, Bio-Rad Laboratories, Redmond, Wash.; ViroSeq HIV-1 Genotyping System with the 3700 Genetic Analyzer, Celera Diagnostics, Alameda, Calif.; HIV 1/2 STAT-PAK ASSAY, Chembio Diagnostic Systems, Inc., Medford, N.Y.; SURE CHECK HIV 1/2 ASSAY, Chembio Diagnostic Systems, Inc., Medford, N.Y.; APTIMA HIV-1 RNA Qualitative Assay, Gen-Probe, Inc., San Diego, Calif.; Home Access HIV-1 Test System, Home Access Health Corp., Hoffman Estates, Ill.; Cambridge Biotech HIV-1 Western Blot Kit, Maxim Biomedical, Inc., Rockville, MD; Maxim Biotech HIV-1 Urine EIA, Maxim Biomedical, Inc., Rockville, Md.; Reveal Rapid HIV-1 Antibody Test, MedMira Laboratories, Inc., Halifax, Nova Scotia, Canada B3S 1B3; UltraQual HIV-1 RT-PCR Assay, National Genetics Institute, Los Angeles, Calif.; OraQuick ADVANCE Rapid HIV-1/2 Antibody Test, OraSure Technologies, Bethlehem, Pa.; OraSure HIV-1 Oral Specimen Collection Device, OraSure Technologies, Bethlehem, Pa.; OraSure HIV-1 Western Blot Kit, OraSure Technologies, Bethlehem, Pa.; Ortho VITROS HIV-1/HIV-2, Ortho-Clinical Diagnostics, Inc, Raritan, N.J.; COBAS AmpliPrep/COBAS TaqMan HIV-1 Test, Roche Molecular Systems, Inc., Pleasanton, Calif.; COBAS Ampliscreen HIV-1 Test18, Roche Molecular Systems, Inc., Pleasanton, Calif.; Roche Amplicor HIV-1 Monitor Test, Roche Molecular Systems, Inc., Pleasanton, Calif.; Fluorognost HIV-1 IFA, Sanochemia Pharmazeutika AG, Vienna, Austria; ADVIA Centaur HIV 1/O/2 Enhanced ReadyPack Reagents, Siemens Healthcare Diagnostics, Inc.; Trugene HIV-1 Genotyping Kit and Open Gene DNA Sequencing System, Siemens Healthcare Diagnostics, Inc.; Versant HIV-1 RNA 3.0 (bDNA), Siemens Healthcare Diagnostics, Inc.; Uni-Gold Recombigen HIV, Trinity Biotech, plc, Bray Co., Wicklow, Ireland.

In one embodiment, the terms “antibody” and “immunoglobulin” are used interchangeably herein. These terms are well understood by those in the field, and refer to a glycosylated (comprising sugar moieties) protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The term “antibody” also includes any protein or peptide containing molecule that comprises at least a portion of an immunoglobulin molecule, such as, but not limited to, one complementarity determining region (CDR) of a heavy chain or light chain constant region, a framework region, or any portion thereof. Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions or classes, e.g., gamma (of about 330 amino acids). The subunit structures and three-dimensional configurations of different classes of immunoglobulins are well known. Any of these embodiments may be used in the present invention.

In one embodiment, an “antigen” includes a full length protein, a derivative of a full-length protein, such as but not limited to, a protein fragment or a synthetic peptide that comprises an amino acid sequence corresponding to a part or parts of a full-length protein, including any modified fragment or synthetic peptide having a ligand attached thereto.

In one embodiment, the stimulating step in the methods of the present invention comprises incubating a second aliquot of the subjects' blood samples in a media comprising an activator of microbe-specific cells. In one embodiment, the stimulating step comprises inducing polyclonal activation of peripheral blood mononuclear cells. In one embodiment, the stimulating step comprises inducing HIV-specific activation of peripheral blood mononuclear cells. In another embodiment, the stimulating step comprises inducing polyclonal activation of lymphocytes. In one embodiment, the virus-specific cells are B-lymphocytes. In another embodiment, the virus-specific cells are T-lymphocytes.

In one embodiment, an activated blood sample comprises both antibodies produced in vivo and antibodies produced by in vitro stimulation.

In one embodiment, an activator is a stimulant. In one embodiment, an aliquot of a tissue sample is stimulated, while in another embodiment, it is activated.

In one embodiment, the activator of the present invention stimulates blood to produce anti-microbial antibodies, while in another embodiment, the activator stimulates blood to secrete anti-microbial antibodies.

In one embodiment, an “activator” for use in the compositions and methods of the present invention is a substance or molecule that induces the activation of a lymphocyte, which is, in one embodiment, is a small lymphocyte, which in one embodiment, is a B cell, a T cell, or a combination thereof in the tissue sample. In one embodiment, the B cells, T cells, or combination thereof, are primed in vivo. In another embodiment, B or T cells are in the “blast” state in the tissue sample, or, in another embodiment, B or T cells are memory cells in the tissue sample. In one embodiment, the substance or molecule is a protein, while in another embodiment, it is a peptide, a nucleic acid molecule, a glycoprotein, etc. In one embodiment, “activation” of cells comprises inducing proliferation of cells, differentiation of cells, enhancement of cellular activity (in one embodiment, antibody production), secretion of various lymphokines and/or cytokines, or a combination thereof.

In one embodiment, the activator for use in the compositions and methods of the present invention activates non-secreting cells, in one embodiment, or not fully activated cells, in another embodiment, or not fully differentiated cells, in another embodiment, or memory cells, in another embodiment.

In one embodiment, the activator is a mitogen. In one embodiment, a “mitogen” is a chemical substance, or a mixture of substances. In one embodiment, a mitogen is one or more proteins, glycoproteins, or a combination of several proteins and glycoproteins with or without other biochemical moieties, that encourages a cell to commence cell division, triggering mitosis. In one embodiment, a mitogen triggers signal transduction pathways in which mitogen-activated protein kinase is involved, leading to mitosis. In one embodiment, mitogens of the present invention are used to induce mitosis in and/or activation of B cells and/or T cells. In one embodiment, mitogens of the present invention are used to induce the formation of antibody secreting blast cells, or plasma cells, from primed differentiating B cells and/or memory B cells.

In one embodiment, the activator of the compositions and methods of the present invention induces the activation of non-secreting B or T cells that are specific for the microbe or virus of interest. In another embodiment, the activator of the present invention induces the expression of viral specific antibodies. In another embodiment, the activator of the present invention induces the transfer from non-secreting B cells to secreting B cells, which in one embodiment, are blasts or plasma cells.

In one embodiment, an activator used in the methods and kits of the present invention enhances blast cell division, which in one embodiment, enhances the production of antibodies and, in another embodiment, enhances differentiation of B cells into plasma cells. In another embodiment, an activator used in the methods and kits of the present invention enhances blast cell division, enhances the production of antibodies, enhances differentiation of B cells into plasma cells, or a combination thereof. In one embodiment, activated B cell blasts secrete antibody and undergo cell division. In one embodiment, plasma cells secrete antibody and do not proliferate.

In one embodiment, viral antigens are used in conjunction with activators to induce activation of non-secreting B cells. Thus, in one embodiment, the compositions of the present invention additionally comprise one or more antigens specific to the virus of interest which, in one embodiment, aids or enhances the transfer from non-secreting B and/or T cells to secreting B and/or T cells, which in one embodiment, are blasts or plasma cells. Similarly, the methods of the present invention may comprise incubating a tissue sample in a media containing a mitogen and one or more viral antigens.

In one related aspect, the activator used in the invention provided herein can be either T-cell dependent or T-cell independent. In one embodiment, the activator used in the compositions and methods of the present invention acts on T-cells, B-cells, or both T cells and B cells. In one related aspect, the activator used to induce activation of non-secreting B cells and the expression of virus specific antibodies is a mitogen, which in one embodiment, is pokeweed mitogen, which in one embodiment, stimulates both B- and T-cells. Other mitogens can be used in practicing the present invention and include, but are not limited to, lectins, such as, concanavalin A, which in one embodiment acts on T cells; bacterial endotoxins, which in one embodiment, is lipopolysaccharide (LPS), which in one embodiment, acts on B cells. In another embodiment, the mitogen is phytohaemagglutinin (PHA), which in one embodiment, acts on T cells. In another embodiment, the mitogen is leucoagglutinin (PHA-L), while in another embodiment, the mitogen is Pisum sativum agglutinin (PSA).

In another embodiment, the activator used in the composition and methods of the present invention is a cytokine, which in one embodiment is a signaling molecule secreted by specific cells of the immune system and glial cells. In one embodiment, said cytokine is an interleukin or interferon. In one embodiment, the cytokine is a lymphokine. In one embodiment, said lymphokine is Interleukin 1, Interleukin 2, Interleukin 3, Interleukin 4, Interleukin 5, Interleukin 6, Interleukin 10, Interleukin 12, Granulocyte-macrophage colony-stimulating factor, Interferon-gamma, or a combination thereof.

In another embodiment, the activator used in the composition and methods of the present invention is a bacterially derived lipid A, a viral-derived peptide, a virus, a biological agent, an anti-immunoglobulin reagent, an antibody against a B and/or T-lymphocyte cellular domain, or a combination thereof. In another embodiment, the activator used in the composition and methods of the present invention is a viral-derived peptide, lectin, bacterial endotoxin, a virus, lipid A, a cytokine, or a lymphokine. In another embodiment, the activator may be a combination of the activators described herein.

In one related aspect, stimulation of cells is achieved by using antibodies against cellular membrane domains. In another embodiment, cells are stimulated by using antibodies against a B-lymphocyte cellular domain, which in one embodiment is a membrane B-lymphocyte cellular domain. In one embodiment, the antibody is anti-IgD, which in one embodiment, is membrane-expressed by: naïve B cells, initially primed B cells, and memory cells. In one embodiment, plasma cells do not express membrane IgD. In one embodiment, primed B cells that have not fully differentiated to plasma cells can be stimulated or activated by contacting them with anti-IgD. In another embodiment, the antibody is anti-IgM. In another embodiment, the antibody is directed against a B cell cellular domain (CD). In another embodiment, the antibody is directed against a T cell CD.

In one embodiment, the membrane B-lymphocyte cellular domain is IgG, IgA, IgE, CD19, or any other membrane structure/domain known in the art. In another embodiment, the membrane B-lymphocyte cellular domain is CD21 or CD81.

In one embodiment, the antibody for use in the methods and compositions of the present invention comprises anti-IgD, anti-IgG, anti-IgA, anti-IgE, or anti-CD19, or anti-CD10, anti-CD23, anti-CD25, and anti-CD40.

In one embodiment, the antibody class used to stimulate a non-secreting cell includes, but is not limited to, an antibody from the IgM, IgG (e.g. IgG1, IgG2, IgG3, IgG4), IgD, IgA, or IgE class.

In another aspect, stimulation of non-secreting B cells, which in one embodiment, are memory cells, to secreting B cells, which in one embodiment, are blasts or plasma cells results in the transformation of the cell to an antibody-secreting blast or plasma cell, whereby the blast or plasma cell secretes antigen-specific antibodies.

In a related aspect, the B lymphocyte of the methods provided herein is a non-secreting B-lymphocytic cell. In another related aspect, the T lymphocyte is a non-secreting T-lymphocytic cell. In yet another related aspect, the activator provided herein activates a non-fully activated B-lymphocytic cell. In another embodiment, the activator activates a non-fully activated T-lymphocytic cell, and in another embodiment the activator activates both T and B cells.

In one embodiment, the incidence determination of the present invention has a low false recent rate. In one embodiment, the false recent rate is under 10%. In another embodiment, the false recent rate is under 5%. In another embodiment, the false recent rate is under 4%. In another embodiment, the false recent rate is under 3%. In another embodiment, the false recent rate is under 2%. In another embodiment, the false recent rate is under 1%.

In another embodiment, the incidence determination of the present invention has a high mean recency duration. In one embodiment, the mean recency duration is approximately a year. In another embodiment, the mean recency duration is approximately 11 months. In another embodiment, the mean recency duration is approximately 10months. In another embodiment, the mean recency duration is approximately 9 months. In another embodiment, the mean recency duration is approximately 8 months. In another embodiment, the mean recency duration is approximately 7 months. In another embodiment, the mean recency duration is approximately 6 months. In another embodiment, the mean recency duration is approximately 5 months. In another embodiment, the mean recency duration is approximately 4 months. In another embodiment, the mean recency duration is approximately 3 months. In another embodiment, the mean recency duration is approximately 2 months. In another embodiment, the mean recency duration is approximately 1 month. In another embodiment, the mean recency duration is approximately 70 days. In another embodiment, the mean recency duration is approximately 60 days. In another embodiment, the mean recency duration is approximately 45 days. In another embodiment, the mean recency duration is approximately 30 days. In another embodiment, the mean recency duration is approximately 14 days. Thus, in one embodiment, an infection will be classified as recent for epidemiological-statistical purposes if it occurred within one of the time frames described hereinabove.

In one embodiment, the SI is calculated only for samples that have undergone seroconversion. In one embodiment, the SI is calculated only for samples in which the unstimulated aliquot (in one embodiment, the first aliquot) gave results above the cut-off of the anti-retroviral antibody assay. In another embodiment, the SI is calculated for all samples, regardless of whether the unstimulated aliquot (in one embodiment, the first aliquot) gave results above the cut-off of the anti-retroviral antibody assay.

In one embodiment, the mean recency of virus infection in said population is determined. According to this aspect and in one embodiment, the statistical recency for each sample in days, weeks, months, or years is calculated based on the ratio of stimulated to unstimulated anti-retroviral antibody levels, and the mean recency of the population is calculated as is known in the art.

In one embodiment, the stimulation index (SI) describes the ratio of stimulated to unstimulated antibody levels. In one embodiment, the mean SI in a population is used to determine the change in incidence of new HIV infections in a population, wherein the mean SI in a population for a specific year is compared to the mean SI for the population in one or more previous years, wherein if the mean SI has increased, it is an indication that there has been an increase in new infections in the population, and wherein if the mean SI has decreased, it is an indication that there has been a decrease in new infections in the population.

In one embodiment, an SI threshold is chosen that provides a low false recent rate. In one embodiment, the SI threshold is 1.5. In another embodiment, the SI threshold is 1.4. In another embodiment, the SI threshold is 1.3. In another embodiment, the SI threshold is 1.2. In another embodiment, the SI threshold is 1.1. In another embodiment, the SI threshold is 1.0. In another embodiment, the SI threshold is 0.95

In one embodiment, a method of the present invention comprises a stimulating step which comprises incubating said sample in a device using any immune-stimulation technology known in the art.

In one embodiment, the device using an immune-stimulation technology is a commercially available tissue culture tube with a special medium which enhances antibody production in vitro in a whole blood sample. As soon as there are, for example, HIV primed B-cells in the blood, (i.e. within days of HIV infection), it is possible to get anti-HIV antibodies produced by them in vitro, at levels detectable by the currently available kits. Current serology measures the levels of HIV-specific antibodies in the blood sample. These levels are of antibodies produced in vivo. Pre-treating the blood sample in the culture tube produces a plasma sample that contains in it, in addition to the antibodies already in the plasma, the antibodies produced in vitro, during the culture step. Antibodies against HIV can be induced in vitro (produced by HIV primed B cells) within days after infection and prior to their appearance/detection in the blood. This enables earlier detection of the infection, using the currently available assays and kits for antibody detection. Thus, using the stimulated plasma as the tested sample gives a better measure of prevalence. Clinical studies have been conducted in several countries around the world showing improved diagnostic sensitivity by using stimulated plasma.

In another embodiment, the present invention provides a kit for determining the incidence of new viral infections in a population comprising: a container for collecting whole blood samples, wherein the container comprises a media comprising an activator of T and/or B cells specific for said virus, an assay for the detection of virus-specific antibodies, and instructions for use.

In another embodiment, the present invention provides a kit for determining the incidence of new viral infections in a population comprising: a container for collecting whole blood samples, wherein the container comprises a media comprising an activator of virus-specific or non-specific lymphocytes, an assay for the detection of virus-specific antibodies, and instructions for use.

In another embodiment, the present invention provides a kit for determining the incidence of new viral infections in a population comprising: a container for collecting whole blood samples, wherein the container comprises a media comprising an activator of virus-specific or non-specific lymphocytes, an assay for the detection of virus-specific antibodies, and instructions for use.

In one embodiment, the assay of the kits of the present invention comprises a means for detection of virus-specific antibodies and non-specific antibodies, as is known in the art.

In another embodiment, the present invention provides a kit for determining the incidence of new HIV infections in a population comprising: a container for collecting whole blood samples, wherein the container contains a media containing an activator of T and/or B cells specific for said HIV, an assay for the detection of HIV-specific antibodies, and instructions for use.

In another embodiment, the present invention provides a kit for determining the incidence of new HIV infections in a population comprising: a container for collecting whole blood samples, wherein the container comprises a media comprising an activator of lymphocytes specific and/or non-specific for said HIV, an assay for the semi-quantification of HIV-specific antibodies, and instructions for use.

In another embodiment, the present invention provides a kit for determining the incidence of pathogenic infections in a population comprising: two containers for collecting tissue samples, wherein one of the containers comprises a media comprising one or more activators of pathogen-specific or non-specific lymphocytes, an assay for the detection of pathogen-specific antibodies, and instructions for use.

In another embodiment, the present invention provides a kit for determining the distribution of recent, non-recent, and late stage pathogenic infections in a population comprising: two containers for collecting tissue samples, wherein one of the containers comprises a media comprising one or more activators of pathogen-specific or non-specific lymphocytes, an assay for the detection of pathogen-specific antibodies, and instructions for use.

In another embodiment, the present invention provides a kit for determining the incidence of human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific antibodies, and instructions for use.

In another embodiment, the present invention provides a kit for determining the distribution of recent, non-recent, and late stage human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific antibodies, and instructions for use.

In one embodiment, the assay in the kits described herein may comprise an assay for the detection of pathogen-specific and non-specific antibodies, where in one embodiment, the pathogen is a virus, a retrovirus, or HIV. In one embodiment, the non-specific antibodies provides a helpful parameter when calculating the SI of the specific antibodies.

In one embodiment, the instructions comprise algorithms for calculating the SI threshold value, etc, as is described herein (e.g. determining the threshold as the mean value of 60% or 70% or 80% or 90% or 95% of the total tested (seropositive) population.

In one embodiment, the assay of the kits of the invention further comprises a means for the detection of non-specific antibodies as a control (e.g. total IgM or total IgG).

In one embodiment, the assay of the kits of the invention further comprises tissue samples from a population of interest, which, in one embodiment, are blood samples.

In one embodiment, kits of the present invention may comprise a packaged combination of reagents in predetermined amounts with instructions for performing a method of the invention. In one embodiment, the kit may comprise suitable reagents for detecting a labeled HIV antigen. For example, when the label is an enzyme, the kit will include substrates and cofactors required by the enzyme (e.g., a substrate precursor which provides the detectable chromophore or fluorophore). In addition, other additives may be included such as stabilizers, buffers and the like. The relative amounts of the various reagents may be varied widely to provide for concentrations in solution of the reagents which substantially optimize the sensitivity of the assay. The reagents may be provided as dry powders, usually lyophilized, including excipients which on dissolution will provide a reagent solution having the appropriate concentration.

HIV antigen for use in kits of the present invention can be provided/obtained from any source known in the art. For example, HIV antigen can be produced using recombinant methods such as those known in the art. Alternatively, HIV antigen can be purchased from a commercial supplier.

In one embodiment, the container of the kit of the present invention is for retaining tissue samples, or in another embodiment, holding, processing, storing, maintaining or collecting tissue samples.

Kits are also provided that are useful as a positive control for the diagnostic assays. For isolation and purification of anti-viral antibodies, the kit can contain viral proteins/antigens coupled to beads (e.g., sepharose beads or nanobeads or other nano-structures). Kits can be provided which contain the antibodies for detection and quantitation of anti-viral antibodies in vitro, e.g. in an ELISA, peptide microarray, bio-chip, or a Western blot. As with the article of manufacture, the kit comprises a container and a label or package insert on or associated with the container. The container holds a composition comprising at least one antigen recognized by the anti-viral antibodies. Additional containers may be included that contain, e.g., diluents and buffers, control antibodies. The label or package insert may provide a description of the composition as well as instructions for the intended in vitro or diagnostic use.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

The method of the present invention includes optionally separating the blood cells from the fluid portion of the blood so that the presence of antibodies, or the presence of antibody-producing cells can be determined. The separation of the blood cells from the fluid portion of the blood can be done by any of several methods well known to those of ordinary skill in the art, including centrifugation or density dependent sedimentation. In one embodiment, the blood cells are not physically separated from the fluid. In another embodiment, peripheral blood mononuclear cells (PBMCs), B-lymphocytes and T-lymphocytes may be separated from the blood prior to culture and assay. Methods of B cell and T cell enrichment are well known in the art and can be carried out by methods that include, but are not limited to, density dependent sedimentation, and/or cell sorting/FACS. After incubation of the tissue with the mitogen, fluid from the top of the blood can easily be extracted and tested for antibody. Optionally, the red blood cells can be lysed either by mild osmotic shock or with a mild detergent. In this way, the white blood cells remain viable. Another method would be to sediment the white blood cells via density, or density gradient.

Generally, the results of a test or assay according to the invention can be presented in any of a variety of formats. The results can be presented in a qualitative fashion. For example, the test report may indicate only whether or not a particular virus-specific antibodies were detected, perhaps also with an indication of the limits of detection. The results may be presented in a semi-quantitative fashion. For example, various ranges may be defined, and the ranges may be assigned a score (e.g., 1+ to 4+) that provides a certain degree of quantitative information. Such a score may reflect various factors, e.g., the number of virus detected, the intensity of the signal (which may indicate the level of expression of virus specific B cells, or T cells), etc. The results may be presented in a quantitative fashion, e.g., as a percentage of cells in which the virus specific antibodies are detected, as a viral specific antibody concentration (as determined via different antibody binding/detection assay), etc. As will be appreciated by one of ordinary skill in the art, the type of output provided by a test will vary depending upon the technical limitations of the test and the biological significance associated with detection.

In one embodiment of the present invention, whole blood is collected in a blood collection tube containing culture medium and mitogen. The blood samples are then incubated with an approximately 1:50-1:500 final dilution of pokeweed mitogen at a concentration of 0.1-2×10⁶ viable cells per ml for four days at 37.degree. C. in a 3-10% CO₂ humidified atmosphere. The blood is then centrifuged and the supernatant fluid is collected and assayed within approximately 24 hours for reactive antibodies by ELISA, lateral flow, and/or blot techniques. In the alternative, an aliquot of fluid may be taken directly from the sample. Each sample should be screened for antibody by lateral flow (Rapid test) or ELISA first, samples considered positive may then be subjected to an additional test, e.g. blot analysis.

In one embodiment, the methods of the present invention comprise the steps described. In another embodiment, the methods of the present invention consist essentially of the steps described. In another embodiment, the methods of the present invention consist of the steps described. In one embodiment, the compositions of the present invention, which in one embodiment, are kits comprise the elements described. In another embodiment, the compositions of the present invention, which in one embodiment, are kits consist essentially of the elements described. In another embodiment, the compositions of the present invention, which in one embodiment, are kits consist of the elements described.

In one embodiment, the methods and kits of the present invention may be used in conjunction with other methods of determining the incidence of retroviral infection known in the art.

### EXAMPLE 1

### Test for Recent HIV Infection Using Stimulation Devices

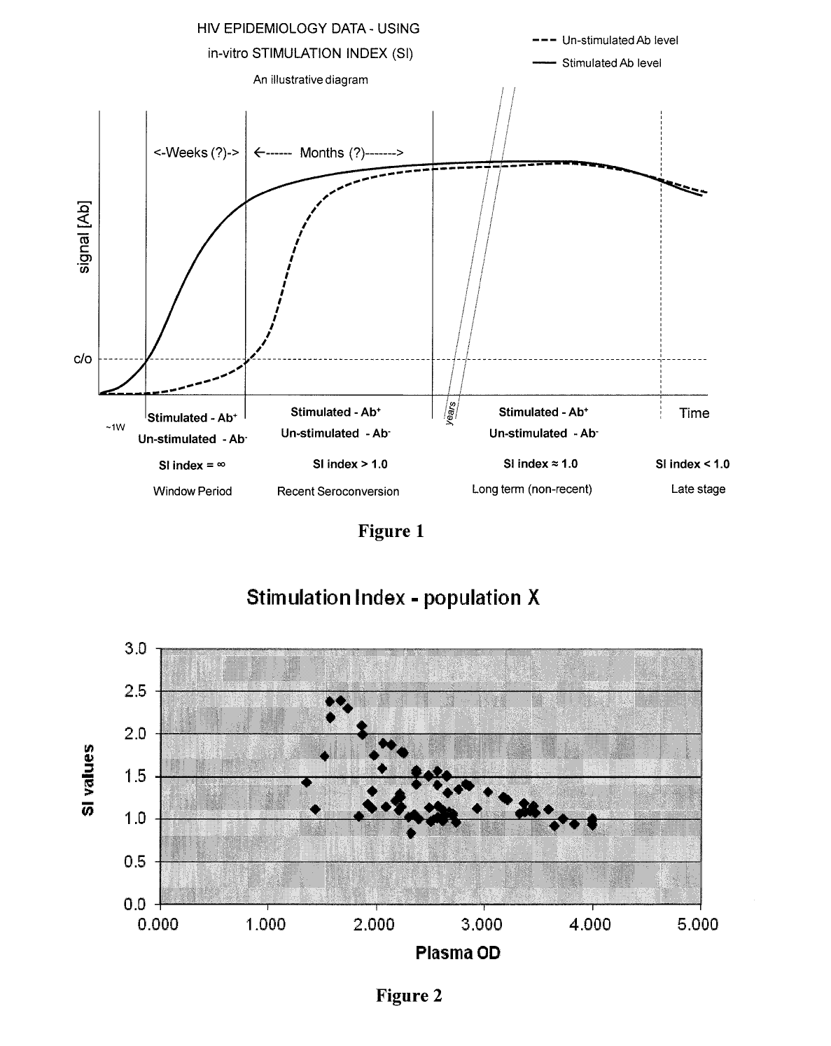

An HIV infection that is in its Seronegative Window Period, namely the period between acquiring the infection and the time of serocoversion at which antibody levels have reached measurable levels, is undetectable by diagnostic tests such as enzyme linked immunosorbent assay (ELISA)/enzyme immunoassay (EIA). To mitigate the effect of this Seronegative Window Period in producing false negative results, stimulation methods and/or stimulation devices were developed to enhance antibody detection when using existing HIV diagnostic tests. The breakthrough stimulation methods and/or stimulation devices stimulates in vivo primed specific immune cells to produce antibodies in vitro, resulting in antibody levels reaching detectable levels sooner after infection, and hence reducing the Seronegative Window Period, as illustrated in FIG. 1.

An unexpected feature of the stimulation methods and/or stimulation devices is that the increased antibody levels in a blood specimen incubated in stimulation methods and/or stimulation devices compared to control blood specimens fades with time after seroconversion (FIG. 1). Comparison of the antibody levels in plasma and stimulated plasma can lead to distinguishing recent seroconversion from older infections, by the increase in antibody levels found in the stimulated plasma (the Stimulation Index). The increased antibody levels at the early stages of seroconversion stem from the fact that the antibody production in vivo is not at full force, and thus additional activation in vitro leads to higher levels of antibodies in the stimulated-plasma. Later on, the immune activation and antibody production are at such high levels in the body that the levels of antibodies measured in the stimulated-plasma do not differ from those in the regular plasma.

Therefore, the Stimulation Index (SI), defined as the ratio of stimulated to unstimulated antibody levels, measured by a semi-quantitative assay may be used as a novel biomarker for testing for recent infection. The potential performance characteristics of a TRI using stimulation methods and/or stimulation devices were investigated, using a dataset captured by the Centers for Disease Control and Prevention (CDC) and the National Institute for the Control of Pharmaceutical and Biological Products in Beijing (NICBPB) on individuals in various regions of China.

Methods: Blood samples in heparin were collected from >350 intravenous drug users (IDUs) in China and brought to the local Center for Disease Control (CDC) laboratory. One ml of blood was transferred to a stimulation method and/or stimulation device within 24 h of collection and incubated for 5 days in 5% CO₂, 37° C. incubator, yielding stimulated-plasma. The remaining plasma was collected and stored. Stimulated-plasma and plasma were run, in parallel, on the same ELISA kit, and O.D. readings were recorded. Plasma from subjects seropositive for HIV were used in the following analyses.

Data: Many samples in this very high risk population, show a Stimulation Index of >1.5 (indicating higher levels of anti HIV antibodies in the stimulated-plasma versus regular plasma, FIG. 2), which is consistent with independent reports of high prevalence and incidence rates in this population.

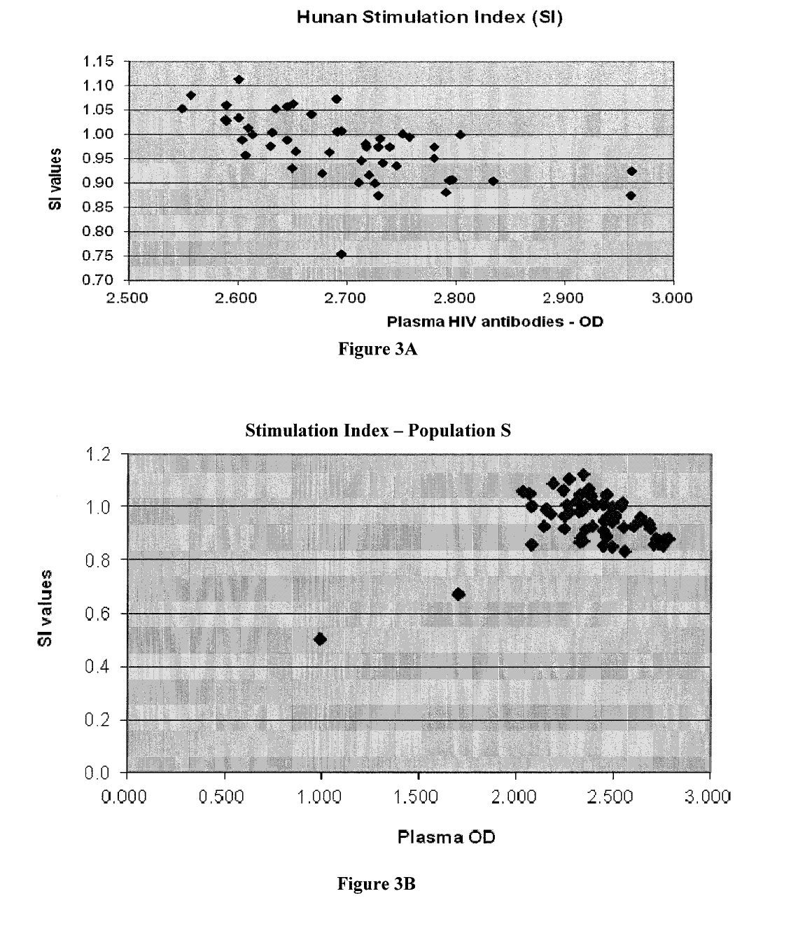

In contrast, in separate populations, none of the samples in either population have a Stimulation Index (SI) of greater than 1.2 (FIG. 3A-B), which is consistent with independent reports describing the infections in this population as being due to medical malpractice 5 years prior to the onset of the study.

### EXAMPLE 2

### Stimulation Index (SI) Distribution

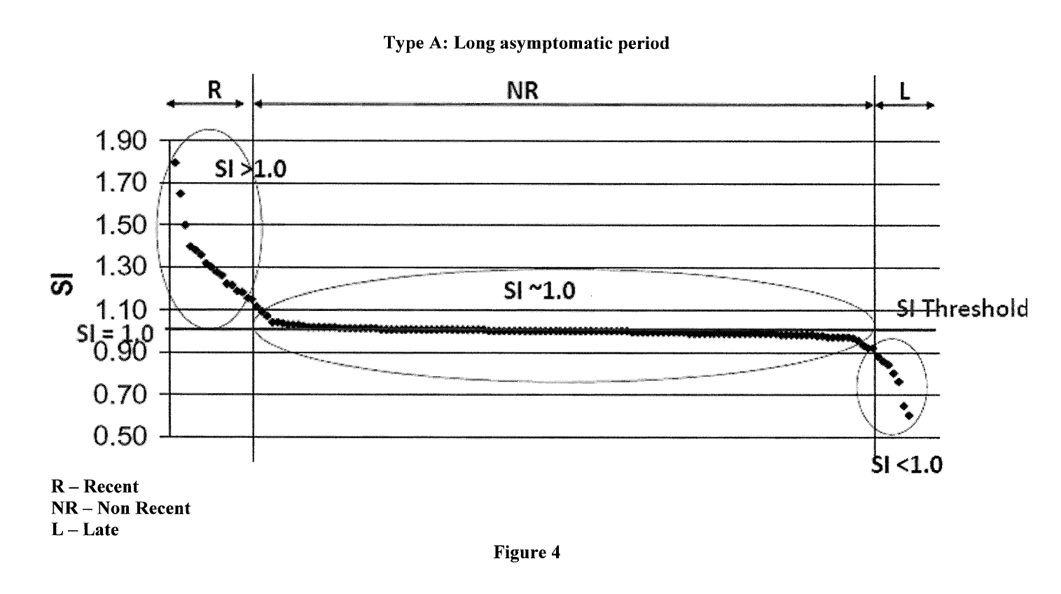

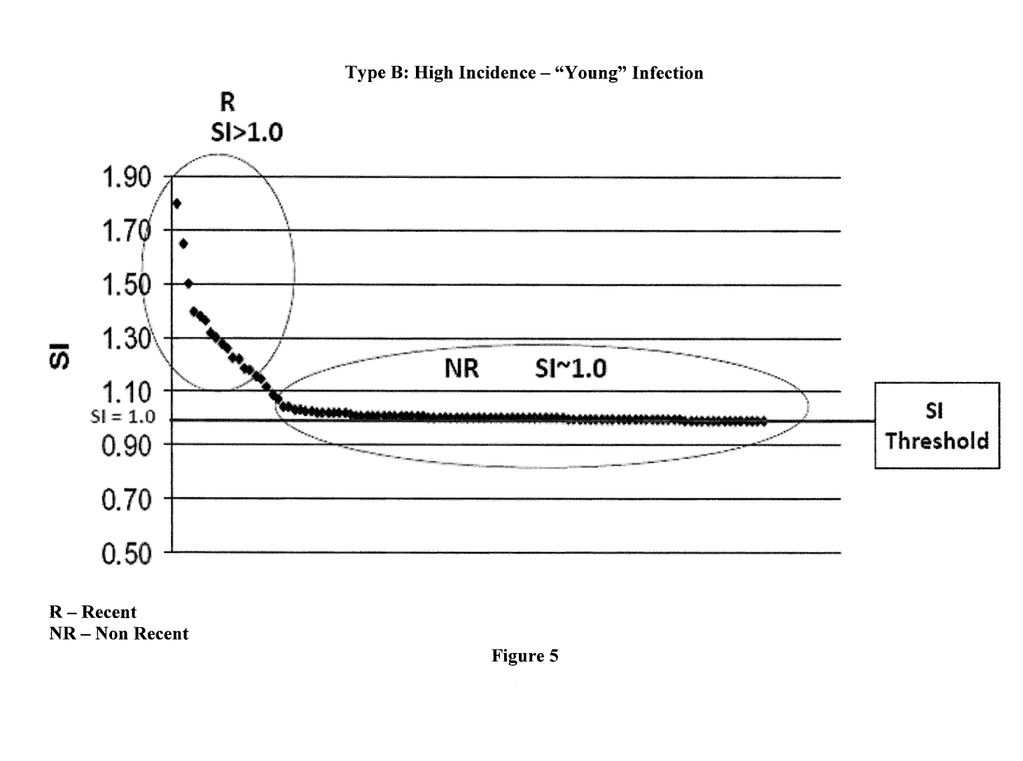

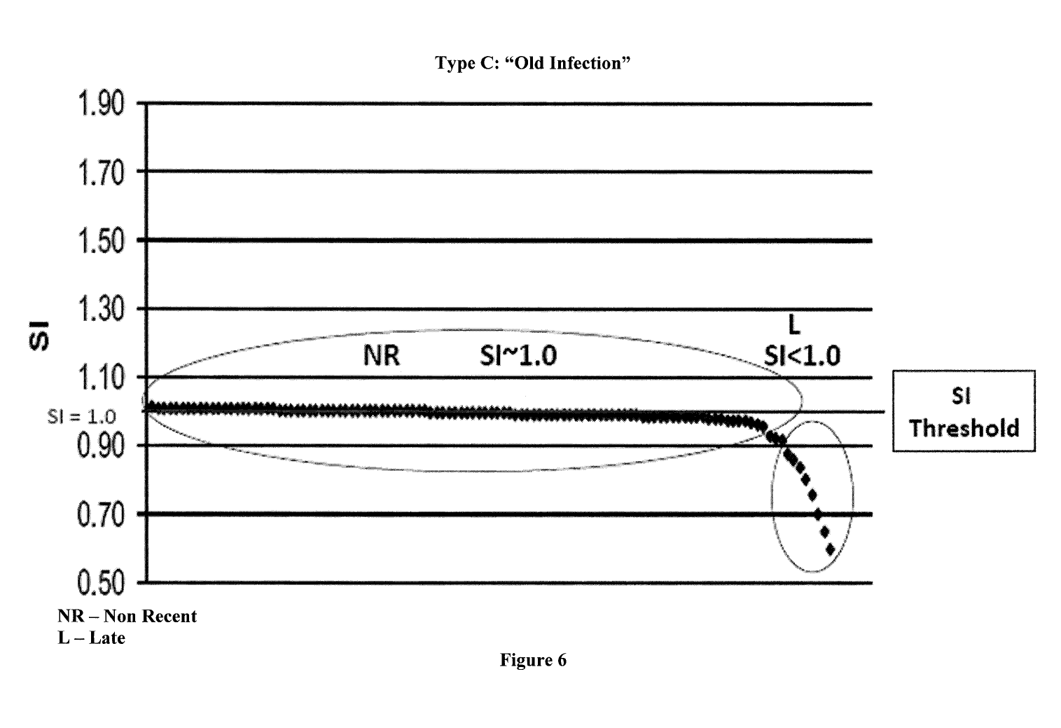

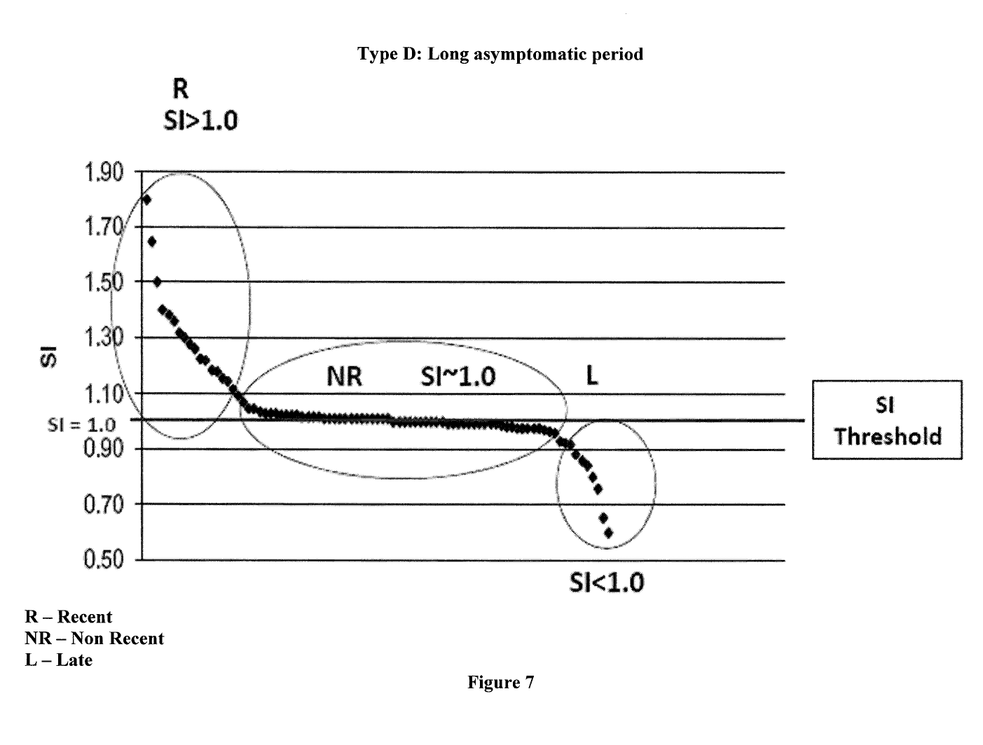

Epidemiological information based on the SIs from various populations demonstrate that infections that have a long asymptomatic period are characterized by an SI distribution as shown in FIG. 4, while infections that have a short asymptomatic period are characterized by an SI distribution as shown in FIG. 7. Populations with a high incidence of recent infections are characterized by an SI distribution skewed toward a larger proportion of high SI values (FIGS. 5), while populations characterized by old, or late stage infections are characterized by a larger proportion of low SI values (FIG. 6).

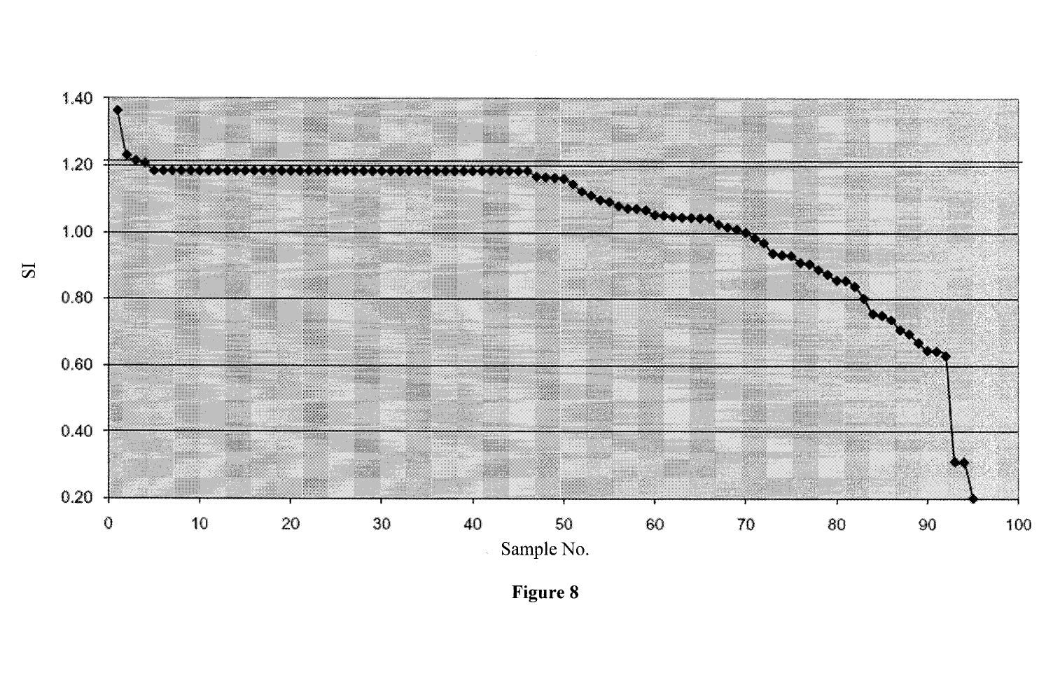

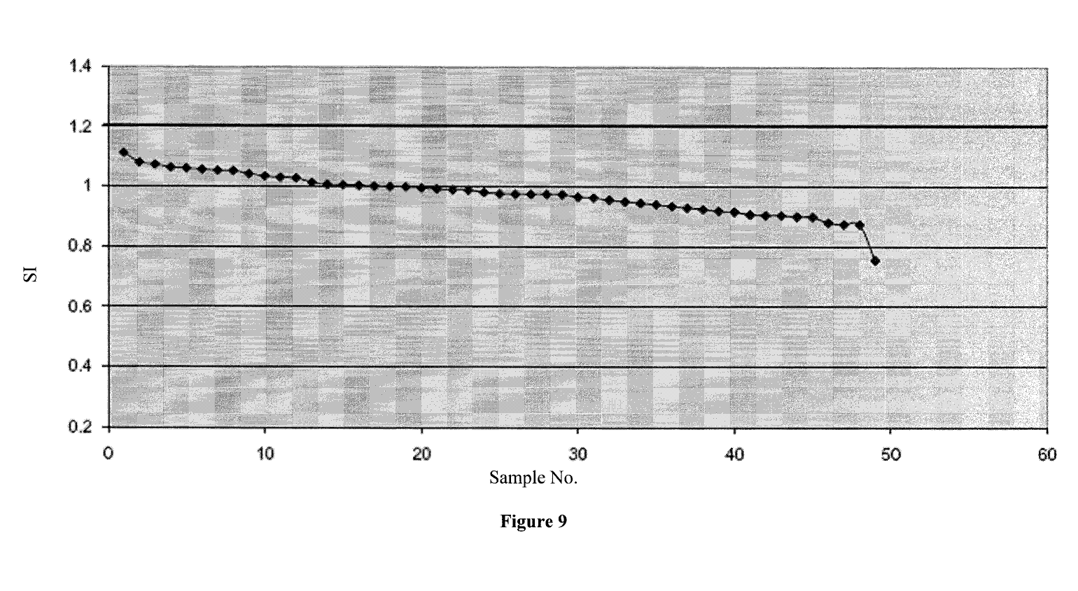

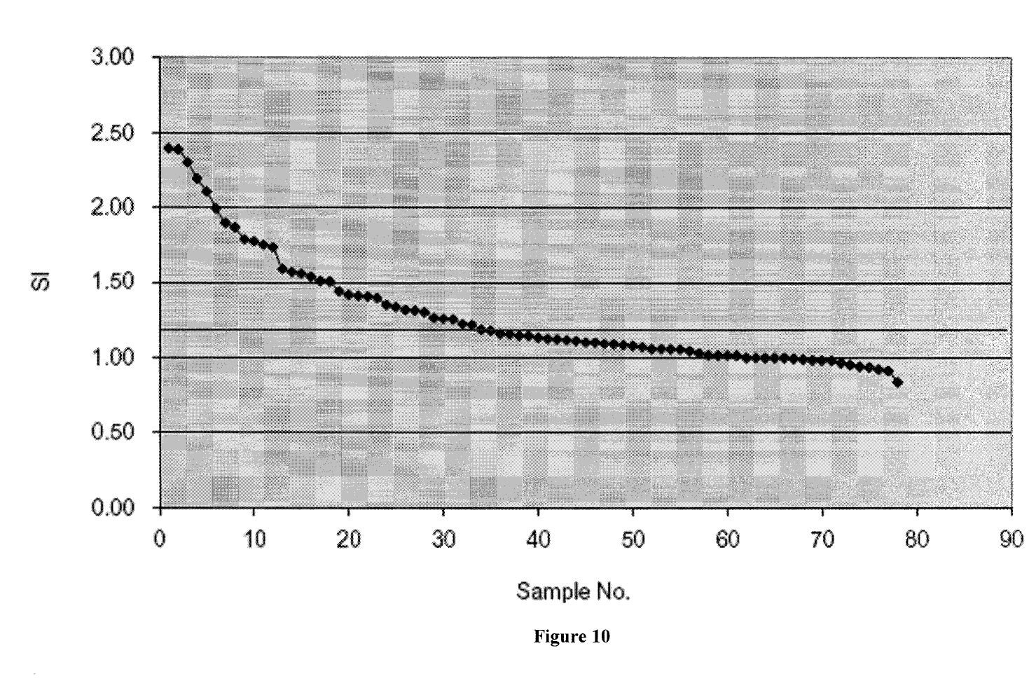

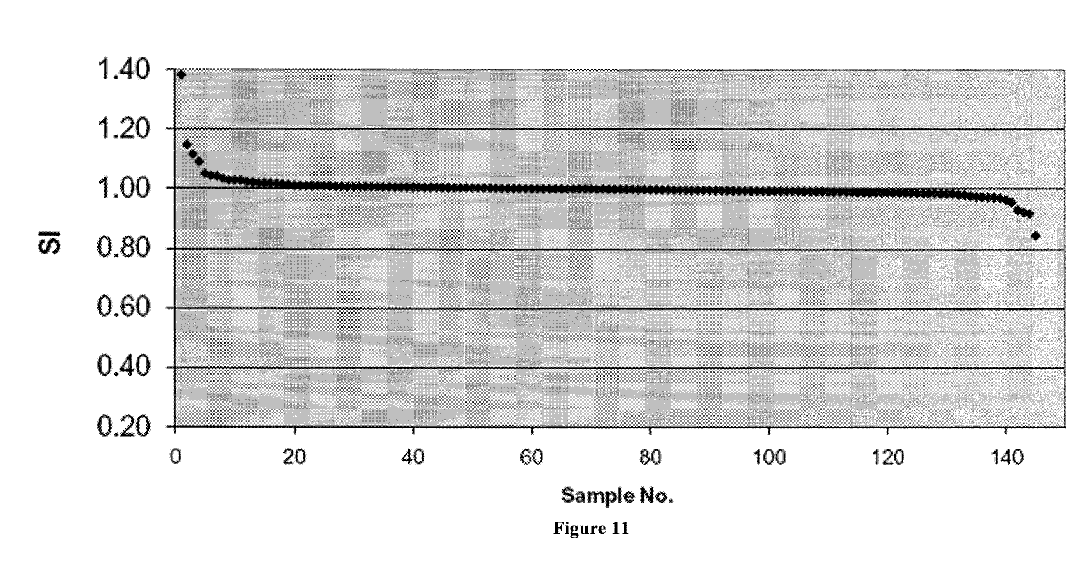

Real-world data supports the models shown in FIGS. 5-6, demonstrating that populations that were known to have a large number of late stage infections have a distribution of SI values as shown in the model of FIG. 6 (FIGS. 8-9), while the distribution of SI values in Chinese and Hungarian populations with a very high incidence rate reflects the model shown in FIG. 5 (FIGS. 10-11).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-56. (canceled)

57. A method of determining the incidence of human immunodeficiency virus (HIV) infections in a population comprising the steps of:
a) obtaining tissue samples from a representative number of subjects in said population;
b) separating a first aliquot of each of said tissue samples for later determination of the initial anti-HIV antibody level;
c) stimulating a second aliquot of each of said tissue samples to produce anti-HIV antibodies in vitro;
d) determining the anti-HIV antibody level in said first and second aliquots of each of said tissue samples;
e) calculating the stimulation index (SI) by comparing the value representing the stimulated anti-HIV antibody level obtained from said second aliquot in step (d) and the value representing the initial anti-HIV antibody level obtained from said first aliquot in step (d) for each sample;
f) determining if the SI obtained in step (e) for each sample is above a pre-determined threshold value, wherein a value below said threshold indicates that the sample was not recently infected and a value above said threshold indicates that the sample was recently infected; and
g) calculating the mean number of recently infected samples divided by the product of the number of samples and the Mean Recency Duration for said threshold,
thereby determining the incidence of HIV infections in said population.

58. The method of claim 57, wherein said stimulating step comprises incubating said tissue samples in step (c) in a media comprising one or more activators of HIV-specific cells, in a media comprising one or more activators of immune cells, or a combination thereof, thereby inducing HIV-specific activation of peripheral blood mononuclear cells, polyclonal activation of peripheral blood mononuclear cells, or a combination thereof.

59. The method of claim 58, wherein said activator is a mitogen, a viral-derived peptide, lectin, bacterial endotoxin, a virus, lipid A, a cytokine, a lymphokine, or a combination thereof.

60. The method of claim 59, wherein said mitogen is phytohaemagglutinin (PHA), concanavalin A (conA), lipopolysaccharide (LPS), pokeweed mitogen (PWM), or a combination thereof.

61. The method of claim 57, wherein said step of determining the anti-HIV antibody level comprises performing an antibody assay on each aliquot of said tissue samples, wherein said antibody assay comprises an enzyme linked immunosorbent assay, a blot, a chemi-luminescence assay, a luminescence assay, or an immunofluorescence assay, a peptide-chip-array, or an antibody chip array.

62. The method of claim 57, wherein the SI is calculated by calculating the ratio of stimulated to non-stimulated anti-HIV antibody levels or the difference between stimulated and non-stimulated anti-HIV antibody levels.

63. The method of claim 57, wherein said tissue samples are whole blood samples.

64. A kit for determining the incidence of human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific antibodies, and instructions for use.

65. The kit of claim 64, wherein said assay comprises an enzyme linked immunosorbent assay, a blot, or an immunofluorescence assay, a chemi-luminescence assay, a luminescence assay, a peptide-chip-array, or an antibody chip array.

66. A method of determining the distribution of recent, non-recent, and late stage human immunodeficiency virus (HIV) infections in a population comprising the steps of:
a) obtaining tissue samples from a representative number of subjects in said population;
b) separating a first aliquot of each of said tissue samples for subsequent determination of the initial anti-HIV antibody level;
c) stimulating a second aliquot of each of said tissue samples to produce anti-HIV antibodies in vitro;
d) determining the anti-HIV antibody level in said first and second aliquots of each of said tissue samples;
e) calculating the stimulation index (SI) by comparing the value representing the stimulated anti-HIV antibody level obtained from said second aliquot in step (c) and the value representing the initial anti-HIV antibody level obtained from said first aliquot in step (c) for each sample;
f) plotting the values of the SI obtained in step (e) for all samples to determine the distribution of recent, non recent, and late-stage infections, wherein samples with an SI value above a pre-determined threshold value have a recent infection, samples with an SI value of approximately said pre-determined threshold value have a non-recent infection, and seropositive samples with an SI value of less than said pre-determined threshold value have a late infection, thereby determining the distribution of recent, non-recent, and late stage HIV infections in said population.

67. The method of claim 66, wherein the SI is calculated by calculating the ratio of stimulated to non-stimulated anti-HIV antibody levels or the difference between stimulated and non-stimulated anti-HIV antibody levels.

68. The method of claim 66, further comprising the step of calculating the ratio of recent, non-recent, and late stage HIV infections to total HIV infections in said population.

69. The method of claim 66, further comprising the step of calculating the area under the curve (AUC) of the plot obtained in step (f), wherein a larger AUC above the threshold indicates more recent HIV infections and a larger AUC under the threshold indicates more late-stage HIV infections.

70. The method of claim 66, wherein said stimulating step comprises incubating said tissue samples in step (c) in a media comprising one or more activators of HIV-specific cells, in a media comprising one or more activators of immune cells, or a combination thereof, thereby inducing HIV-specific activation of peripheral blood mononuclear cells, polyclonal activation of peripheral blood mononuclear cells, or a combination thereof.

71. The method of claim 70, wherein said activator is a mitogen, a viral-derived peptide, lectin, bacterial endotoxin, a virus, lipid A, a cytokine, a lymphokine, or a combination thereof.

72. The method of claim 71, wherein said mitogen is phytohaemagglutinin (PHA), concanavalin A (conA), lipopolysaccharide (LPS), pokeweed mitogen (PWM), or a combination thereof.

73. The method of claim 66, wherein said step of determining the anti-HIV antibody level comprises performing an antibody assay on each aliquot of said tissue samples, wherein said antibody assay comprises an enzyme linked immunosorbent assay, a blot, a chemi-luminescence assay, a luminescence assay, or an immunofluorescence assay, a peptide-chip-array, or an antibody chip array.

74. The method of claim 66, wherein said tissue samples are whole blood samples.

75. A kit for determining the distribution of recent, non-recent, and late stage human immunodeficiency virus (HIV) infections in a population comprising: two containers for collecting whole blood samples, wherein one of the containers comprises a media comprising one or more activators of HIV-specific or non-specific lymphocytes, an assay for the detection of HIV-specific antibodies, and instructions for use.

76. The kit of claim 75, wherein said assay comprises an enzyme linked immunosorbent assay, a blot, or an immunofluorescence assay, a chemi-luminescence assay, a luminescence assay, a peptide-chip-array, or an antibody chip array.
